(19) United States
(12) Patent Application Publication
(10) Pub. No.: US 2023/0233518 A1
(43) Pub. Date: Jul. 27, 2023

(54) CATECHIN CONTAINING COMPOSITIONS AND USES

(71) Applicants: ADAMAS BIOTECH S.R.L, Maglie (LE) (IT); DTECH S.R.L., Maglie (LE) (IT); TEA SOLUTIONS, HARA OFFICE INC., Tokyo (JP)

(72) Inventors: Piero CHIARELLI, Maglie (LE) (IT); Antonio MORINI, Maglie (LE) (IT); Saverio BETTUZZI, Parma (IT); Hirofumi TACHIBANA, Tokyo (JP); Yukihiko HARA, Tokyo (JP)

(21) Appl. No.: 17/918,733

(22) PCT Filed: Apr. 14, 2021
(86) PCT No.: PCT/IT2021/050111

§ 371 Date: Oct. 13, 2022

(30) Foreign Application Priority Data
Apr. 14, 2020 (IT) 102020000007816

(51) Int. Cl. A61K 31/353; A61P 31/14; A61K 45/06; A61K 9/00

(52) U.S. Cl. CPC A61K 31/353; A61P 31/14; A61K 45/06; A61K 9/006

(57) ABSTRACT

The invention relates to a composition containing catechins found in green tea, to pharmaceutical compositions and medical devices comprising the composition and to the use of these compositions and medical devices in the treatment of coronavirus infections, especially infection with SARS-CoV-2.

The present invention relates to a composition for use inter alia in the treatment or prevention of coronavirus infection. In particular, the invention relates to a catechin-containing composition and to a pharmaceutical composition comprising the catechin-containing composition for use in the treatment of coronavirus infection, especially infection with SARS-CoV-2. The invention also relates to a medical device comprising two components, one component of which comprises the catechin-containing composition and to its use in the treatment or prevention of coronavirus infection, especially infection with SARS-CoV-2.

### BACKGROUND

Coronaviruses are RNA viruses which cause respiratory infections in mammals and birds. In humans they are responsible for a range of diseases, including some cases of the common cold, as well as more serious respiratory infections, including SARS (severe acute respiratory syndrome), MERS (Middle-East respiratory syndrome) and COVID-19. Human coronaviruses are zoonotic pathogens, many of which are thought to have their origins in bats (Forni et al, January 2017).

COVID-19 disease is caused by the coronavirus SARS-CoV-2, which originated in Wuhan, China in December 2019 and which was responsible for a global pandemic in 2020 and 2021. The SARS-CoV-2 virus is highly transmissible and spreads when an infected person exhales virus-containing, usually in respiratory droplets or aerosols. The particles are emitted when the infected person coughs or sneezes or even sings, talks or breathes and are able to enter and infect another person via the mucous membranes of the mouth, nose or eyes.

The symptoms of SARS-CoV-2 infection vary greatly from person to person (Grant et al, June 2020), with some patients being completely asymptomatic, while others suffer from severe illness and death. Common symptoms include headache, loss of smell and taste, nasal congestion and rhinorrhea, cough, muscle pain, sore throat, fever, diarrhoea and breathing difficulties. No proven therapy is currently available for mild or moderate cases of COVID-19, other than supportive care.

Green tea is a good source of catechins which have been found to exhibit powerful antioxidant, antiviral and anti-inflammatory properties (Ohishi et al, 2016). Green tea catechins are also known to exhibit immunological effects. For example, the polyphenol epigallocatechin-3-gallate (EGCG) found in green tea is known to downregulate TLR4 signalling (Byun et al, 2010) and inhibit peptidoglycan-induced TLR2 signalling (Byun et al, 2011). It has also been found that certain green tea extracts containing catechins and caffeine were able to enhance the effect of a split influenza virus vaccine (Won et al, 2016).

Patients infected with coronaviruses who develop pneumonia often die as a result of complications related to an over-active cytokine-mediated inflammatory response, known as a cytokine storm (Mehta et al, 2020). Several studies have identified IL-6 levels as prognostic for severe disease in COVID-19 positive patients at presentation (Henry et al, 2020).

Mhatre et al suggested green and black tea polyphenols as possible candidates for COVID-19 treatment and performed a number of molecular docking studies (Mhatre et al, 17 Jul. 2020). However, although several catechin compounds found in tea, including epigallocatechin-3-gallate (EGCG), were considered to have potential, no studies were conducted on COVID-19 patients. It is also clear that the polyphenol compounds found in green and black teas interact with a number of different biological systems and it is therefore impossible to tell without conducting in vivo studies, whether these compounds, if used to treat COVID-19, would have side effects associated with such interactions. Finally, the authors note that EGCG is unstable when consumed orally and has low bioavailability and suggest that it may be preferable to use structural derivatives rather than EGCG itself.

There has also been speculation as to whether EGCG might have a protective effect in diseases which lead to a cytokine storm, including COVID-19 (Menegazzi et al, 2020). These authors note that EGCG has been proposed for use in the treatment of autoimmune conditions as diverse as rheumatoid arthritis, Sjogren's syndrome, multiple sclerosis and inflammatory bowel diseases and that polyphenols present in green tea extracts have been shown to have anti-inflammatory and anti-fibrotic effect. The authors further speculate that EGCG and green tea extracts may be of use in treating sepsis and may have antiviral activity. They note that green tea extracts and EGCG supplementation could be advantageous in COVID-19 because of their multitarget action but also remark that there is no direct evidence that EGCG treatment could improve COVID-19 outcome and suggest that a clinical trial would be needed to establish this. It is also worth noting that these authors describe the many and varied biological activities of green tea extracts and of EGCG in particular. This gives rise to the possibility that any therapy based on green tea extracts could result in side effects. Again, this cannot be tested except in a clinical trial.

The present inventors have carried out a number of tests and have found that catechins found in green tea are surprisingly effective in the treatment of infection with SARS-CoV-2 and COVID-19 disease. The inventors have also found that there appear to be few side effects associated with the treatment. They have also developed pharmaceutical compositions comprising green tea catechins and have established an effective dosage regimen.

### SUMMARY OF THE INVENTION

In a first aspect of the present invention there is provided a catechin-containing composition comprising a catechin selected from epigallocatechin-3-gallate (EGCG), epigallocatechin (EGC), epicatechin-3-gallate (ECG) and epicatechin (EC) or a mixture thereof for use in the treatment or prevention of coronavirus infection.

In a second aspect of the invention, there is provided a pharmaceutical composition for use in the treatment or prevention of coronavirus infection, wherein the pharmaceutical composition comprises a catechin-containing composition of the first aspect.

In a third aspect of the invention, there is provided a medical device, in particular one which is adapted for administration to the oral or nasal mucosa, and which comprises a first component and a second component, characterized in that:

the first component is an aqueous gel which comprises a biocompatible polymer, a polyacid and/or salt thereof, a preservative and a catechin-containing composition of the first aspect; and
the second component is an aqueous solution comprising a salt of a bivalent, trivalent or multivalent cation, or a combination of such salts.

### DETAILED DESCRIPTION OF THE INVENTION

In the present specification, the “dry weight” of a composition refers to the total mass of the composition excluding the mass of any solvent which may be included in the composition.

In the present specification, the term “aqueous gel” refers to any water-based gel, such as a hydrogel, i.e. a network of crosslinked polymer chains in an aqueous dispersion medium.

In the present specification, the term “multivalent cation” refers to a cation with a valency higher than three. For example, a multivalent cation may have a valency of four or five.

In the present specification, the terms “prophylaxis” and “prevention” are used interchangeably.

The terms “chemical environment which is hostile to coronaviruses” and “chemical environment having anti-coronavirus activity”, when used to describe the film formed by the medical device of the third aspect of the invention, refer to an environment comprising one or more compounds having pharmacological activity against coronaviruses. An example of such an environment is an aqueous gel, suitably a hydrogel, which comprises the catechin-containing composition of the first aspect of the invention, specifically an aqueous gel, suitably a hydrogel, which comprises a catechin selected from epigallocatechin-3-gallate (EGCG), epigallocatechin (EGC), epicatechin-3-gallate (ECG) and epicatechin (EC) or a mixture thereof.

In a first aspect, the invention relates to a catechin-containing composition comprising a catechin selected from epigallocatechin-3-gallate (EGCG), epigallocatechin (EGC), epicatechin-3-gallate (ECG) and epicatechin (EC) or a mixture thereof for use in the treatment or prevention of coronavirus infection. In particular, the catechin-containing composition is for use in the treatment or prevention of SARS-CoV-2 infection.

These catechins are typically found in tea extracts, particularly green tea extracts and therefore the catechin-containing composition may be a green tea extract. The catechin-containing composition is suitably a solid, dry composition which does not comprise a solvent.

The catechin-containing composition suitably comprises epigallocatechin-3-gallate (EGCG). The catechin-containing composition suitably comprises a mixture of at least two of the catechins listed above. In particular, the catechin-containing composition suitably comprises epigallocatechin-3-gallate (EGCG) and epigallocatechin (EGC).

Most suitably, the catechin-containing composition comprises all of the above catechins, i.e. the catechin composition is a mixture comprising EGCG, EGC, ECG and EC.

The catechin-containing composition may comprise catechins in an amount of 75% to 98% by weight, suitably 80% to 98% by weight, more suitably 85% to 95% by weight and typically about 90% by weight, with respect to the dry weight of the composition. In addition to EGCG, EGC, ECG and EC, the catechin-containing composition may comprise further catechins, for example gallocatechin gallate (GCG) and/or catechin, for example (+/−) catechin (DL-C).

Suitably, the catechin-containing composition comprises EGCG in an amount of 50% to 80% by weight, more suitably 55% to 75% by weight or 56% to 72% by weight, for example about 62% to 75% by weight, with respect to the dry weight of the composition.

Some suitable catechin-containing composition comprise EGC in an amount of 1% to 30% by weight, more suitably 2 to 25% by weight, for example 5% to 22% or 5% to 20% by weight, with respect to the dry weight of the composition. In other cases, the amount of EGC may be from about 1% to 10% by weight, for example 2% to 9% by weight of the dry weight of the composition.

The catechin-containing composition may comprise ECG in an amount of 1 to 10% by weight, for example 2 to 10% by weight or 4 to 10% by weight with respect to the dry weight of the composition.

The catechin-containing composition may comprise EC in an amount of 1 to 15% by weight. In some suitable compositions, the amount of EC may be from about 1% to 5% by weight with respect to the dry weight of the composition. In other suitable compositions, the amount of EC may be from about 7% to 15% by weight with respect to the dry weight of the composition.

Where GCG is present, it may be present in an amount of up to 8% e.g. up to 5% e.g. 0.1-8% e.g. 0.1-5% e.g. 0.1-2% by weight with respect to the dry weight of the composition.

Where DL-C is present, it may be present in an amount of up to 3% e.g. up to 2% e.g. 0.1-3% e.g. 0.1-2% e.g. 0.5-1.5% by weight with respect to the dry weight of the composition.

It is preferred that the catechin-containing composition should contain, at most, minimal amounts of non-catechin compounds which occur in tea extracts, for example compounds such as caffeine, theobromine and gallic acid. A typical catechin-containing composition for use according to the invention is preferably substantially free of caffeine, theobromine and gallic acid. For example, the catechin-containing composition may comprise less than 1% by weight (e.g. 0 to 1% by weight) caffeine and/or less than 1% by weight (e.g. 0 to 1% by weight) theobromine and/or less than 1% by weight (e.g. 0 to 1% by weight) gallic acid, where % by weight is expressed with respect to the dry weight of the composition. Suitably, the amount of caffeine present in the catechin containing composition is less than 0.5% by weight (e.g. 0 to 0.5% by weight), with respect to the dry weight of the composition. In some cases, it is not possible to remove the caffeine, theobromine and/or gallic acid completely and so trace amounts may remain. Therefore, the catechin-containing composition for use according to the invention may comprise 0.0001% to 1% by weight caffeine and/or 0.0001% to 1% by weight and/or 0.0001% to 1% by weight gallic acid, where the % by weight is with respect to the dry weight of the composition.

A particularly suitable catechin-containing composition for use in the present invention is sold under the trade mark Theaphenon® E, available from Tea Solutions, Hara Office, Inc, Tokyo, Japan.

The invention further provides a method for the treatment or prevention of coronavirus infection, the method comprising administering to a patient in need of such treatment and effective amount of a catechin-containing composition comprising a catechin selected from epigallocatechin-3-gallate (EGCG), epigallocatechin (EGC), epicatechin-3-gallate (ECG) and epicatechin (EC).

In addition, the invention provides the use of a composition comprising a catechin selected from epigallocatechin-3-gallate (EGCG), epigallocatechin (EGC), epicatechin-3-gallate (ECG) and epicatechin (EC) in the manufacture of a medicament for the treatment or prevention of coronavirus infection.

Particularly suitable features of the method and the use are as described above for the pharmaceutical composition.

The catechin-containing composition may be administered in the form of a pharmaceutical composition and therefore, in a further aspect of the invention there is provided a pharmaceutical composition for use in the treatment or prevention of coronavirus infection, wherein the pharmaceutical composition comprises a catechin-containing composition according to the first aspect of the invention.

The pharmaceutical composition may be formulated for administration by any route but suitably is formulated for oral administration; for administration by inhalation; or for administration to the oral or nasal mucosa.

Formulations for oral administration in the present invention may be presented as: discrete units such as capsules, sachets or tablets each containing a predetermined amount of the catechin-containing composition; as a powder or granules; as a solution or a suspension of the catechin-containing composition in an aqueous liquid or a non-aqueous liquid; or as an oil-in-water liquid emulsion or a water in oil liquid emulsion; or as a bolus etc. Liquid formulations may, in some cases, be provided in the form of drinks, which may be provided in containers adapted to provide a single dose of the catechin-containing composition.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate, stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the catechin-containing composition in a free-flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the catechin-containing composition.

Other formulations suitable for oral administration include lozenges comprising the catechin-containing composition in a flavoured base, usually sucrose and acacia or tragacanth; pastilles comprising the catechin-containing composition in an inert base such as gelatin and glycerin, or sucrose and acacia; and mouthwashes comprising the catechin-containing composition in a suitable liquid carrier.

Particularly suitable oral formulations include discrete units such as capsules, tablets and sachets, especially tablets and capsules and more especially capsules.

Inhaled administration, i.e. topical administration to the lungs, may be achieved by use of a non-pressurised formulation such as an aqueous solution or suspension. These may be administered by means of a nebuliser e.g. one that can be hand-held and portable or for home or hospital use (ie non-portable). The formulation may comprise excipients such as water, buffers, tonicity adjusting agents, pH adjusting agents, surfactants and co-solvents.

Alternatively, topical administration to the lung may be achieved by use of an aerosol formulation. Aerosol formulations typically comprise the catechin-containing composition suspended or dissolved in a suitable aerosol propellant, such as a chlorofluorocarbon (CFC) or a hydrofluorocarbon (HFC). Suitable CFC propellants include trichloromonofluoromethane (propellant 11), dichlorotetrafluoromethane (propellant 114), and dichlorodifluoromethane (propellant 12). Suitable HFC propellants include tetrafluoroethane (HFC-134a) and heptafluoropropane (HFC-227). The propellant typically comprises 40%-99.5% e.g. 40%-90% by weight of the total inhalation composition. The formulation may comprise excipients including co-solvents (e.g. ethanol) and surfactants (e.g. lecithin, sorbitan trioleate and the like). Other possible excipients include polyethylene glycol, polyvinylpyrrolidone, glycerine and the like. Aerosol formulations are packaged in canisters and a suitable dose is delivered by means of a metering valve which meters a specified volume (e.g. as supplied by Bespak, Valois or 3M or alternatively by Aptar, Coster or Vari).

Suspension liquid and aerosol formulations (whether pressurised or unpressurised) will typically contain the compound of the invention in finely divided form, for example with a D₅₀ of 0.5-10 μm e.g. around 1-5 μm. Particle size distributions may be represented using D₁₀, D₅₀ and D₉₀ values. The D₅₀ median value of particle size distributions is defined as the particle size in microns that divides the distribution in half. The measurement derived from laser diffraction is more accurately described as a volume distribution, and consequently the D₅₀ value obtained using this procedure is more meaningfully referred to as a Dv₅₀ value (median for a volume distribution). As used herein Dv values refer to particle size distributions measured using laser diffraction. Similarly, D₁₀ and D₉₀ values, used in the context of laser diffraction, are taken to mean Dv₁₀ and Dv₉₀ values and refer to the particle size whereby 10% of the distribution lies below the D₁₀ value, and 90% of the distribution lies below the D₉₀ value, respectively.

Particularly suitable inhaled formulations include formulations adapted for inhalation via a nebuliser.

The pharmaceutical compositions of the invention may be administered to a patient in an amount such that the dose of the catechin-containing composition according to the invention is from 10 mg to 3000 mg per day e.g. 10 mg to 2000 mg per day e.g. 10 mg to 1000 mg per day.

Orally administered pharmaceutical compositions of the invention may be administered to a patient in an amount such that the dose of the catechin-containing composition according to the invention is from 300 mg to 3000 mg per day e.g. from 600 mg to 2000 mg per day. In some cases, the oral dose may be from 600 mg to 1800 mg or from 600 mg to 1000 mg per day. Alternatively, the oral dose may be from 900 to 1800 mg/day e.g. 900 to 1500 mg/day. Alternatively, the oral dose may be from 100 mg to 2000 mg per day e.g. from 100 mg to 1000 mg per day e.g. from 300 mg to 1000 mg per day.

Suitably, the oral dose of the catechin-containing composition is such that the oral dose of EGCG is less than 800 mg per day.

Pharmaceutical compositions of the invention which are administered by inhalation, especially inhalation via a nebuliser may be administered to a patient in an amount such that the dose of the catechin-containing composition is from 10 mg to 60 mg per day, more suitably from 25 mg to 35 mg per day.

In some cases, a catechin-containing composition for use in the treatment or prevention of coronavirus infection, for example SARS-CoV-2 infection, may be administered by two separate routes, for example orally and by inhalation. In this case two separate pharmaceutical compositions may be administered to the patient, for example an oral formulation and a formulation adapted for topical administration to the lung as described above.

The catechin-containing composition may be administered to the patient orally in an amount of from 300 mg to 2000 mg per day, suitably from 600 mg to 1800 mg or 600 mg to 1000 mg per day or, alternatively, from 900 mg to 1800 mg per day; and by inhalation via a nebuliser in an amount of from 10 mg to 60 mg per day, suitably from 25 mg to 35 mg per day.

The present invention further provides a method for the treatment or prevention of coronavirus infection, for example SARS-CoV-2 infection comprising administering to a patient in need of such treatment a pharmaceutical composition as defined above in an amount such that the dose of the catechin-containing composition is from 10 mg to 3000 mg per day e.g. 10 mg to 2000 mg per day e.g. 10 mg to 1000 mg per day.

The invention also provides the use of a pharmaceutical composition as defined above in the manufacture of a medicament for the treatment or prevention of coronavirus infection, wherein the pharmaceutical composition is administered to a patient in an amount such that the dose of the catechin-containing composition is from 10 mg to 3000 mg per day e.g. 10 mg to 2000 mg per day e.g. 10 mg to 1000 mg per day.

Suitable doses for oral administration and administration by inhalation are as set out above. As noted above, oral administration may be as a discrete dosage form such as capsules, tablets or sachets, especially capsules or tablets and more especially capsules. Administration by inhalation is suitably via a nebuliser.

The invention also provides a method for the treatment or prevention of coronavirus infection, for example SARS-CoV-2 infection, comprising administering to a patient in need of such treatment a catechin-containing composition according to the present invention, wherein the catechin-containing composition is administered to the patient orally in an amount of from 300 mg to 2000 mg per day, suitably from 600 mg to 1800 mg or 600 mg to 1000 mg per day or, alternatively, from 900 mg to 1800 mg per day; and by inhalation via a nebuliser in an amount of from 10 mg to 60 mg per day, suitably from 25 mg to 35 mg per day.

The invention further provides the use of a catechin-containing composition as defined above in the manufacture of a medicament for the treatment or prevention of coronavirus infection, wherein the catechin-containing composition is administered to the patient orally in an amount of from 300 mg to 2000 mg per day, suitably from 600 mg to 1800 mg or 600 mg to 1000 mg per day or, alternatively, from 900 mg to 1800 mg per day; and by inhalation via a nebuliser in an amount of from 10 mg to 60 mg per day, suitably from 25 mg to 35 mg per day.

As noted above, oral administration may be as a discrete dosage form such as capsules, tablets or sachets, especially capsules or tablets and more especially capsules. Administration by inhalation is suitably via a nebuliser.

In a further aspect of the invention, the pharmaceutical composition comprising the catechin-containing composition may be adapted for administration to the oral or nasal mucosa.

In some cases, the pharmaceutical composition is provided as a part of a component of a novel medical device, which itself forms a further aspect of the invention.

The medical device of the invention comprises at least two components and is adapted to form in situ i.e. at the site of its application a film of solid gel containing an intermolecular fluid at its interior.

The film both represents an effective physical barrier against the spread of coronaviruses such as SARS-CoV-2 and has at its interior a chemical environment that is hostile to such coronaviruses. This hostile environment is provided by the catechin-containing composition which, as demonstrated in the examples below, has been shown to reduce SARS-CoV-2 infection in patients.

The way in which the protective film/barrier is formed in situ is such that it is perfectly adapted to the shape and surface of the body mucosa, thus ensuring that the said film performs its purpose with ease and continuity.

The medical device of the invention comprises at least a first component which is in aqueous gel form, and which is suitably a hydrogel, and at least a second component, a cross-linking component which is an aqueous solution of a salt. The first component also comprises a biologically active substance, which is a catechin-containing composition according to the first aspect of the invention. The first component is sometimes herein referred to as the “base gel”.

Therefore, in a further aspect of the invention there is provided a medical device, in particular one which is suitable for or adapted for administration to the oral or nasal mucosa, and which comprises a first component and a second component, characterized in that: the first component is an aqueous gel which comprises a biocompatible polymer, a polyacid and/or salt thereof, a preservative and a catechin-containing composition comprising a catechin selected from epigallocatechin-3-gallate (EGCG), epigallocatechin (EGC), epicatechin-3-gallate (ECG) and epicatechin (EC) or a mixture thereof; and the second component is an aqueous solution comprising a salt of a bivalent, trivalent or multivalent cation, or a combination of such salts.

The preventive application of the two component medical device of this aspect of the invention is intended to the oral and/or nasal mucous membranes of a patient who requires protection against coronavirus infection, especially infection with SARS-CoV-2. Suitably the application will uniformly cover the mucous membranes to which it is applied.

Two component products comprising a hydrogel and a cross-linking solution are known, for example from U.S. Pat. No. 5,147,648, WO 2014/181299 and WO 2019/180530. However, none of these documents teaches or suggests a medical device which is suitable for the treatment or prevention of coronavirus infection and in which the hydrogel component comprises a catechin compound.

The first component may comprise at least one biocompatible polymer e.g. it may comprise more than one biocompatible polymer. The first component may comprise at least one polyacid and/or salt thereof e.g. it may comprise more than one polyacid and/or salt thereof. The first component may comprise at least one preservative e.g. it may comprise more than one preservative. The second component may comprise at least one salt of a bivalent, trivalent or multivalent cation e.g. it may comprise more than one salt of a bivalent, trivalent or multivalent cation.

The first component, i.e. the aqueous gel, which is viscous fluid, and the second component may be separately applied to the oral and/or nasal mucous membranes of the patient. Mixing of the first and second components results in the formation in situ of a rubbery protective film. This film forms a physical barrier which prevents access of coronavirus (e.g. SARS-CoV-2) particles to the mucous membranes. The interior of the film remains in an aqueous gel state and provides a chemical environment which has anti-coronavirus activity.

Suitably, the first component is applied first to the oral and/or mucous membranes of the patient and the second component is applied subsequently.

In the medical device of the invention, the polymer of the first component adheres to the mucous membranes and brings the surface of the mucous membranes into close contact with the polymer chains of the bioadhesive system which is formed. The adhesion occurs through the forming of secondary chemical cross-linking bonds between the polymer chains of the protective film and the mucous membrane. Hydrogen bonds and van der Waals forces may also play a part. This gives rise to stable adhesion of the product on the surface of the mucous membrane to which treatment is applied.

The application of the medical device therefore gives rise to an effective and stable bioadhesive barrier which blocks coronavirus infections, specifically SARS-CoV-2 infections, both mechanically (i.e. as a physical barrier) and chemically (by the antiviral activity of the catechin-containing composition). Furthermore, because the barrier is formed in situ, it conforms exactly to the contours of the mucous membrane to which it is applied, ensuring a continuous protective barrier over the surface of the mucous membrane.

The medical device of the invention also ensures the formation of an elastic film having sufficient porosity to allow the passage of gases, especially oxygen and carbon dioxide, while preventing the passage of coronaviruses such as SARS-CoV-2.

It is also possible that the product could be used in the treatment or prevention of other viral infections, for example influenza, HIV and HBV infections

In a further aspect of the invention, there is provided a bioadhesive system, more specifically a bioadhesive film situated at the oral and/or nasal mucous membranes of a patient and obtainable by the application of the medical device of the invention to the oral and/or nasal mucous membranes of a patient.

The first and second components may be applied to the oral and/or nasal mucosa of the patient by means of a spray, typically a nebulised spray.

Suitably, the bioadhesive film is obtainable by first applying the first component of the medical device to the oral and/or nasal mucosa of the patient and subsequently applying the second component. The characteristics of the bioadhesive film are as described above.

As noted above, the medical device comprises a first component, which is in the form of an aqueous gel, for example a hydrogel, and a second, cross-linking component, which is an aqueous solution.

Suitably, in the first component of the medical device, the biocompatible polymer is polyvinyl alcohol (PVA) having a molecular weight of 10 to 1,000,000, suitably with a molecular weight greater than 10,000, i.e. in the range of 10,000 to 1,000,000. The PVA is suitably present in the first component in an amount of 0.001 to 30% by weight, more suitably 1% to 4% by weight of the first component.

In the first component, the polyacid or salt thereof is sodium alginate, wherein the sodium alginate is present in an amount of 0.001% to 5% by weight of the first component, for example about 0.5% by weight. The viscosity of the sodium alginate is suitably from 50 to 5000 cP at 20° C. e.g. from 50 to 4000 cP at 20° C. e.g. from 50 to 2000 cP at 20° C. Viscosity is suitably measured using a rotating viscosimeter.

Suitably, the first component of medical device comprises 0.01% to 5% by weight, more suitably 0.1 to 0.5% by weight of EGCG, EGC, ECG, EC or a mixture thereof.

The EGCG, EGC, ECG, EC or mixture thereof may be provided in a catechin-containing composition according to the first aspect of the invention and the amount of the catechin-containing composition in the first component may be calculated to provide EGCG, EGC, ECG, EC or a mixture thereof in an amount of 0.01% to 5% by weight, more suitably 0.1 to 0.5% by weight relative to the weight of the first component.

The first component of the medical device may further comprise an anti-inflammatory agent and/or an antiviral agent.

Suitable anti-inflammatory agents include acetylsalicylic acid, flurbiprofen or combinations thereof. The anti-inflammatory agent may be present in an amount of 0.001% to 30% by weight and suitably from 0.5 to 5% by weight with respect to the weight of the first component.

Suitable anti-viral agents include (2S)-2-{(2R,3S,4R,5R)-[5-(4-Aminopyrrolo[2,1-f] [1,2,4] triazin-7-yl)-5-cyano-3,4-dihydroxy-tetrahydrofuran-2-ylmethoxy]phenoxy-(S)-phosphorylamino} propionic acid 2-ethyl-butyl ester (commercially known as remdesivir). The antiviral agent may be present in the first component in an amount of 0.001% to 30%, suitably 0.1% to 5% by weight with relative to the weight of the first component.

Suitable preservatives for inclusion in the first component include parabens, propyl paraoxybenzoate and methyl paraoxybenzoate or any combination thereof.

The first component may comprise a buffer and/or a stabilizer. For example, buffers are weak acid buffers which stabilize the pH between 4 and 6, in particular those based on ascorbate salts. The first component may thus comprise, as a stabilizer and antioxidant protecting the activity of the catechins, an ascorbic acid-sodium ascorbate buffer, which buffer stabilizes the pH between 4 and 6.

The second component of the medical device is an aqueous salt solution in which the cation of the salt is bivalent, trivalent or has higher valency (i.e. is multivalent). The salt may be a chloride or an iodide and may be, for example, calcium chloride, magnesium chloride or zinc chloride. The concentration of the salt in the aqueous solution of the second component may be from 0.001 molar to the saturation concentration.

In the medical device of the invention, the first and second components are suitably to be administered in the form of a spray, suitably a nebulised spray.

Suitably, the first and second components of the medical device are provided in separate first and second containers, each of said separate containers being provided with a nebuliser/spray system. It is preferred that each of the separate containers is provided with means for providing a metered volume of each of the first and second components. Suitable means includes a metering valve which can meter a specified volume. The metered volume may be from 0.05 mL to 0.5 mL. As noted above, the amount of the catechin-containing composition in the first component is calculated to provide EGCG, EGC, ECG, EC or a mixture thereof in an amount of 0.01% to 5% by weight, more suitably 0.1 to 0.5% by weight relative to the weight of the first component. Therefore, each metered volume of spray may provide from 0.005 mg to 25 mg of EGCG, EGC, ECG, EC or a mixture thereof, more suitably 0.05 mg to 2.5 mg of EGCG, EGC, ECG, EC or a mixture thereof. The nebuliser/spray system may include an applicator or mouthpiece for applying the liquid to the oral or nasal mucosa.

Suitably, the total daily dose of EGCG, EGC, ECG, EC or a mixture thereof is about 5 mg to 30 mg, and the spray may be administered several times per day, for example three times per day in doses of 1.67 g to 10 mg or four times a day in doses of 1.2 mg to 7.5 mg EGCG, EGC, ECG, EC or a mixture thereof. The number of sprays to be applied will depend upon the volume of the metered volume but, for example, the first component may be administered four times a day with four sprays for each administration (e.g. two sprays for each nostril or one spray for each nostril and two sprays for the mouth).

The second component is suitably administered immediately after the first component. The metered volume of the second component may also be from 0.05 mL to 0.5 mL and the recommended volume of the second component may be the same as or different from that of the first component.

The medical device of the invention may be provided as a kit. Therefore, in a further aspect of the invention there is provided a kit comprising a first container containing the first component of the medical device, and a second container containing the second component of the medical device.

In the kit of the invention, each of the first and second containers is suitably provided with a nebuliser/spray system. The nebuliser/spray system may provide a metered volume of each of the first and second components as described above for the medical device.

Other suitable features of the kit are also as set out above for the medical device.

As noted above, the medical device is particularly suitable for the treatment or prevention, and especially for the prevention, of coronavirus infection, especially infection with SARS-CoV-2.

Therefore, in a further aspect of the invention, there is provided a medical device or a kit as described above for use in the treatment or prevention of coronavirus infection, especially SARS-CoV-2 infection.

The invention further provides a method for the treatment or prevention of coronavirus infection, especially SARS-CoV-2 infection, the method comprising administering to a patient in need of such treatment or prevention an effective amount of components of a medical device of the invention or administering to a patient in need of such treatment or prevention an effective amount of the components of a kit according to the invention.

There is also provided the use of a medical device or a kit as described above, and components thereof, in the manufacture of a medicament for the treatment or prevention of coronavirus infection, especially SARS-CoV-2 infection.

Particularly suitable features of the use and the method are as detailed above for the product and the kit.

In further aspects of the invention, there is provided a catechin-containing composition as defined for the first aspect or a pharmaceutical composition as defined for the second aspect for use in the treatment or prevention of lung injury in a patient. The lung injury may be caused by coronavirus (e.g. SARS-CoV-2) infection or may, for example, be caused by asthma, chronic obstructive pulmonary disease or fibrosis.

Further aspects include a method for the treatment or prevention of lung injury (e.g. caused by coronavirus (e.g. SARS-CoV-2) infection or by asthma, chronic obstructive pulmonary disease or fibrosis), the method comprising administering to a patient in need of such treatment and effective amount of a catechin-containing composition (or a pharmaceutical composition comprising such composition) comprising a catechin selected from epigallocatechin-3-gallate (EGCG), epigallocatechin (EGC), epicatechin-3-gallate (ECG) and epicatechin (EC). In addition, the invention provides the use of a composition comprising a catechin selected from epigallocatechin-3-gallate (EGCG), epigallocatechin (EGC), epicatechin-3-gallate (ECG) and epicatechin (EC) (or a pharmaceutical composition comprising such composition) in the manufacture of a medicament for the treatment or prevention of lung injury (e.g. caused by coronavirus (e.g. SARS-CoV-2) infection or by asthma, chronic obstructive pulmonary disease or fibrosis).

The invention may also be defined by the numbered clauses set out below.

1. A medical device comprising at least a first component and at least a second component characterized in that said first component is an aqueous gel comprising at least one biocompatible polymer, at least one polyacid and/or salt thereof, at least one preservative, and at least one substance, which is biochemically active against the spread of coronaviruses, while said second component is an aqueous solution comprising at least one salt of a bivalent, trivalent or multivalent cation, or combinations thereof.
2. The medical device according to the previous claim, wherein the substance biochemically active against the spread of coronaviruses is chosen from catechins, polyphenols and flavonoids.
3. The medical device according to any of claims 1, 2 wherein the first component further comprises an anti-inflammatory and/or an antiviral.
4. The medical device according to any of the preceding claims wherein the biocompatible polymer present in the first component is polyvinyl alcohol with a molecular weight in the range of 10 to 1,000,000, and in a concentration with respect to said first component of between 0.001-30% w/w, while the polyacid is sodium alginate, which is present in a concentration of between 0.001-5% w/w with respect to said first component, and the preservative belongs either to the class of parabens or the group of propyl paraoxybenzoate and methyl paraoxybenzoate, or any combination thereof.
5. The medical device according to the preceding claim, wherein the polyvinyl alcohol has a molecular weight greater than 10,000, and a concentration with respect to the said first component between 1%-4% w/w, while the sodium alginate is present in a concentration of 0.5% w/w with respect to said first component, and has a viscosity between 50 and 2,000 cp at 20° C.
6. The medical device according to any of the preceding claims wherein the substances biochemically active against the spread of coronaviruses are chosen from epicatechin, epigallocatechin, epicatechin 3-gallate and epigallocatechin 3-gallate, or combinations thereof.
7. The medical device according to any of claims 3-6 wherein the anti-inflammatory is chosen from acetylsalicylic acid, flurbiprofen, or combinations thereof, and has a concentration with respect to said first component between 0.001-30% w/w.
8. The medical device according to the preceding claim, wherein the anti-inflammatory has a concentration with respect to said first component between 0.5-5% w/w.
9. The medical device according to any of claims 3-8 wherein the antiviral is (2S)-2-{(2R, 3S, 4R, 5R)-[5-(4-Aminopyrrolo[2,1-f][1,2,4] triazine-7-yl)-5-cyano-3, 4-dihydroxy-tetrahydro-furan-2-ylmethoxy] phenoxy-(S)-phosphorylamino} propionic acid 2-ethyl-butyl ester in a concentration with respect to said first component of between 0.001-30% p/p.
10. The medical device according to the preceding claim, wherein the antiviral has a concentration between 0.1-5% w/w with respect to said first component.
11. The medical device according to any of the preceding claims, wherein the first component comprises, as a stabilizer and antioxidant protecting the activity of the substances biochemically active against the spread of coronaviruses, the ascorbic acid-sodium ascorbate buffer, which buffer stabilizes the pH between 4 and 6.
12. The medical device according to any of the preceding claims wherein the second component is an aqueous salt solution of a salt selected from:
chlorides and iodides.
13. The medical device according to the preceding claim wherein the salt is selected from calcium chloride, magnesium chloride, and zinc chloride in a concentration between 0.001 molar and the saturation concentration of the solution.
14. The medical device according to any of the preceding claims, wherein the first component comprises polyvinyl alcohol having a molecular weight of about 90,000 in a concentration of about 1% w/w, sodium alginate in a concentration of about 0.5% w/w, and the second component is represented by an aqueous calcium chloride salt solution having a concentration of between 0.001 and 10 M.
15. A process for preparing a medical device comprising at least a first component and at least a second component, wherein said first component is obtained by:
dissolving polyvinyl alcohol with a molecular weight of 90,000 in water at a concentration of 1% w/w;
stirring the solution, until a homogeneous solution is obtained;
adding high-molecular-weight sodium alginate to the concentration of 0.5% w/w;
adding sodium ascorbate in a concentration of 0.2% w/w;
adding a mixture of epicatechin, epigallocatechin, epicatechin-3-gallate and epigallocatechin-3-gallate in a concentration of 0.3% w/w;
   and wherein the second component is obtained by preparing an aqueous solution of 1M calcium chloride.
   16. The process according to the preceding claim, wherein in the preparation of the first component, the addition of acetylsalicylic acid is additionally provided at a concentration of 5% by weight.
   17. The medical device as defined in any of claims 1-14 for use in a method for prophylactic and therapeutic treatment of diseases caused by coronavirus.
   18. The medical device as defined in any of claims 1-14 for use in a method of prophylactic and therapeutic treatment of Covid-19, wherein said medical device acts as a precursor to a film capable of acting as a static mechanical virus inhibitor of SARS-COV-2, and as an agent against the spread of coronaviruses.
   19. The medical device as defined in any of claims 1-14 for use in a method of prophylactic and therapeutic treatment of diseases caused by HIV.
   20. The medical device as defined in any of claims 1-14 for use in a method for prophylactic and therapeutic treatment of diseases caused by HBV.
   21. The medical device as defined in any of claims 1-14 for use according to claims 17-20, wherein both the first component and the second component are to be administered in the form of a nebulized spray.
   22. A kit comprising at least one container suitable for containing the first component of the medical device defined in any of claims 1-14, and at least one second container suitable for containing the second component of said medical device, said first and second containers being provided with a nebulizer/spray system.
   23. The kit according to the preceding claim for use in a method for the treatment of diseases caused by coronavirus.
   24. The kit according to claim 22 for use in a method for prophylactic and therapeutic treatment of Covid-19.
   25. The kit according to claim 22 for use in a method for prophylactic and therapeutic treatment of diseases caused by HIV.
   26. The kit according to claim 22 for use in a method for prophylactic and therapeutic treatment of diseases caused by HBV.

The invention also provides a medical device, in particular one which is suitable for or adapted for administration to the oral or nasal mucosa, and which comprises a first component and a second component, characterized in that:

the first component is an aqueous gel which comprises a biocompatible polymer, a polyacid and/or salt thereof, a preservative, and a catechin selected from epigallocatechin-3-gallate (EGCG), epigallocatechin (EGC), epicatechin-3-gallate (ECG) and epicatechin (EC) or a mixture thereof; and
the second component is an aqueous solution comprising a salt of a bivalent, trivalent or multivalent cation, or a combination of such salts.

The invention also provides a medical product, in particular one which is suitable for adapted for administration to the oral or nasal mucosa, and which comprises a first component and a second component, characterized in that:

the first component is an aqueous gel which comprises a biocompatible polymer, a polyacid and/or salt thereof, a preservative, and a catechin selected from epigallocatechin-3-gallate (EGCG), epigallocatechin (EGC), epicatechin-3-gallate (ECG) and epicatechin (EC) or a mixture thereof; and
the second component is an aqueous solution comprising a salt of a bivalent, trivalent or multivalent cation, or a combination of such salts.

The invention also provides a two component system, in particular one which is suitable for adapted for administration to the oral or nasal mucosa, and which comprises a first component and a second component, characterized in that:

the first component is an aqueous gel which comprises a biocompatible polymer, a polyacid and/or salt thereof, a preservative, and a catechin selected from epigallocatechin-3-gallate (EGCG), epigallocatechin (EGC), epicatechin-3-gallate (ECG) and epicatechin (EC) or a mixture thereof; and
the second component is an aqueous solution comprising a salt of a bivalent, trivalent or multivalent cation, or a combination of such salts.

The suitable features of the components of the respective components of the just-mentioned medical device, medical product and two component system are as described above for other aspects of the invention. Suitably the catechin is provided in a catechin containing composition which comprises a mixture of catechins and e.g. comprises epigallocatechin-3-gallate (EGCG), epigallocatechin (EGC), epicatechin-3-gallate (ECG) and epicatechin (EC).

The invention will now be described in greater detail with reference to the Examples. In the Examples, the catechin-containing composition Theaphenon® E was used. Theaphenon® E (ThE) is a dried extract of green tea (Camellia sinensis) which contains approximately 40-50 parts leaves to 1 part extract. ThE is available from Tea Solutions, Hara Office, Inc, Tokyo, Japan. ThE comprises a number of catechins, including EGCG. The composition varies from batch to batch but ThE comprises total catechins in an amount of 85% to 95% by weight with respect to the dry weight of ThE. The total content of EGCG is from 56% to 72% by weight with respect to the dry weight of ThE, with the remainder of the catechin content being made up of other catechins, particularly EGC but also ECG and EC. ThE also contains not more than 1.0% by weight caffeine, not more than 1.0% by weight theobromine and not more than 0.5% by weight gallic acid, where weight percentages are with respect to the dry weight of ThE.

In Examples 1 and 2 and lung injury and inflammation were induced using lipopolysaccharide (LPS).

### Example 1—Effect of ThE on Cell Numbers in Bronchoalveolar Lavage Fluid (BALF) in LPS-Induced Lung Injury in a Mouse Model

### Materials and Methods

6-week old male C57BL6J mice (Kyudo, Saga, Japan) were fed with MF Diet (KBT Oriental, Saga, Japan) and treated with oral administration of ThE having the composition set out in Table 1 (Tea Solutions, Hara Office, Inc, Tokyo, Japan) dissolved in endotoxin free water (Otsuka, Tokushima, Japan) and received the single intratracheal administration of LPS (0.05 mg/kg; Sigma Aldrich) dissolved in endotoxin free water (Otsuka). After 24 hours, mice were sacrificed under isoflurane vapour (Fujifilm, Tokyo, Japan) and BALF were harvested. Cell numbers were evaluated by hematacytometer. The results are shown in Table 2.

TABLE 1
Composition of ThE used in Examples 1 and 2

| Component | | % by weight |
|---|---|---|
| Total Catechins | | 94.87 |
| of which: | EGC (7.91%) | |
| | EC (2.63%) | |
| | DL-C (1.15%) | |
| | EGCG (73.92%) | |
| | GCG (7.34%) | |
| | ECG (1.92%) | |
| Caffeine | | 0.13 |
| Theobromine | | Trace |
| Gallic Acid | | Trace |

TABLE 2
Cell Numbers in BALF

| | | | ThE, 20.29 mg/kg |
|---|---|---|---|
| | Negative | | containing |
| | Control | Control | EGCG 15 mg/kg |
| Cell numbers | 20.33 ± 3.17 | 155.63 ± 19.41 | 79.29 ± 8.81 |
| (×10⁴ cells/mL) | | | |

Dunnett’s test for results for negative control and ThE: P < 0.001 compared with control.

LPS treatment elicited cell increase in BALF and LPS-elicited upregulation of cell numbers were attenuated by ThE treatment. BALF increase is an indicator for acute lung injury and therefore the results demonstrate that ThE has a protective effect against acute lung injury. Administration of ThE reduced damage to the lungs following LPS administration by about 50% as demonstrated by a 50% reduction of the cells released after injury.

In summary, the results demonstrate that lung injury caused experimentally by intratracheal administration was markedly inhibited by oral administration of ThE. This is an indication that ThE is likely to be effective in treating or preventing lung injury e.g. caused by SARS-CoV-2 infection.

### Example 2—Effect of Oral Administration of ThE in LPS-Induced Systemic Inflammation in a Mouse Model

### Materials and Methods

6-week old male C57BL6J mice (Kyudo, Saga, Japan) were fed with MF Diet (KBT Oriental, Saga, Japan) and treated with oral administration of ThE having the composition set out in Table 1 (Tea Solutions, Hara Office, Inc, Tokyo, Japan) dissolved in endotoxin free water (Otsuka, Tokushima, Japan) and received the single intraperitoneal (i.p.) injection of LPS (15 mg/kg; Sigma Aldrich) dissolved in endotoxin free water (Otsuka). After 6 hours, mice were sacrificed under isoflurane vapour (Fujifilm, Tokyo, Japan) and serum were harvested. Inflammatory cytokines were evaluated using ELISA kit (R and D, Minneapolis, Minn., USA) in accordance with the manufacturer's protocol. The results are shown in Table 3.

LPS treatment elicited upregulation of inflammatory cytokine levels in plasma. This LPS-elicited upregulation was attenuated by ThE treatment. The upregulation of inflammatory cytokine levels is a critical factor in cytokine storm and septic shock and ThE appears to have a protective effect against acute lung injury.

IL-6 is a key driver of cytokine storm, which is known to occur in the most seriously ill COVID-19 patients. Therefore, these results indicate that ThE is likely to be effective in the treatment of SARS-CoV-2 infection, and the prevention of lung injury resulting therefrom, and, indeed, in the prevention or treatment of acute lung injury arising from other causes. The utility of ThE in treating SARS-CoV-2 infection was confirmed by the trial described in Example 3 below.

TABLE 3
Inflammatory Cytokines in Serum

| | | | ThE, 135.27 mg/kg |
|---|---|---|---|
| | Negative | | containing |
| | Control | Control | EGCG 100 mg/kg |
| IL-6 (pg/mL) | 0.00 | 171765.23 ± 10010.68 | 93575.64 ± 19816.67 |
| MCP1 (pg/mL) | 0.00 | 36057.74 ± 4041.68 | 17184.00 ± 5183.38 |

Dunnett’s test for results for negative control: P < 0.001 compared with control.
Dunnett’s test for results for ThE: P < 0.05 compared with control.

### Example 3—Efficacy of a Green Tea Extract for Treatment of Patients with SARS-CoV-2 Infection (COVID-19 Syndrome)

The composition of the ThE powder used in the trial is set out in Table 4 and the product formula for the ThE capsules is set out in Table 5.

TABLE 4
Composition of ThE used in Clinical Trial

| Component | | % by weight |
|---|---|---|
| Total Catechins | | 90.34 |
| of which: | EGC (19.58%) | |
| | EC (1.63%) | |
| | EGCG (64.04%) | |
| | GCG (0.42%) | |
| | ECG (4.67%) | |
| Caffeine | | 0.14 |
| Theobromine | | Trace |
| Gallic Acid | | Trace |

TABLE 5
Product Formula of ThE Capsules used in Clinical Trial

| Component | Quantity per Capsule |
|---|---|
| Fill Material | |
| Theaphenon ® E (see Table 4) | 300 mg |
| Corn Starch | 58 mg |
| Microcrystalline cellulose | 18 mg |
| Calcium stearate | 4 mg |
| Shell Material | |
| Hard Capsule (HPMC, clear, capsule | 70 mg |
| size: No 1 | |

Before testing the efficacy of ThE on COVID-19 patients, we tested safety and tolerability of inhalation and capsules administration in 2 healthy volunteers (SB and his wife, GM). Both were negative for COVID-19, but affected by symptoms of influenza. The treatment was well tolerated and the flu symptoms disappeared rapidly. One volunteer with COVID-19 symptoms was treated at home by the study doctor SC early in the pandemic. Later, SC was badly exposed to the virus when her personal protective equipment failed while treating a violently-ill COVID-19 patient in the ward. She used the treatment as described here, had no adverse effects and never got sick.

### Description of Clinical Trial

Inclusion criteria: adults over 18 years, positive to nasopharyngeal swab for COVID-19, symptomatic. Symptoms: low-grade fever and/or loss of taste and/or smell and/or respiratory or gastrointestinal symptoms. Exclusion criteria: none.

Primary objectives: symptoms remission, disease regression, patient recovery; Secondary objectives: reduction of markers of inflammation and time to switch to negative SARS-COV-2 swab.

Patients had to be symptomatic to receive a nasopharyngeal swab test for SARS-Cov-2. According to the rules in force during the lockdown, they were visited by the family doctors at home (LG and SC) within a few days (about 5) when their symptoms worsened.

All ten patients received, discussed and signed the Informed Consent with SC. They received standard of care throughout. The clinical conditions at the doctor's first visit, when first nasopharyngeal swab test was performed and before ThE administration, are shown in Table 6, along with patient demographics, co-morbidities and other medications. No patients received any anti-viral medication along the study trial.

At recruitment day (TO) the doctor (SC) took the first blood sample. Blood samples were sent to different laboratories in the territory for standard analyses. Later, when patients' symptoms were gone (a median of 9.2 days later, range 7-15), SC took a second blood sample (T1). Analytes included: blood count, hemoglobin, CRP (C-reactive protein), ESR (erythrocyte sedimentation rate), alpha-1 anti-trypsin (AAT), interleukin 6 (IL-6), and other indicators, as shown in Table 7. At recruitment (T0) therapy began (capsules plus inhalation) and lasted for 15 days. Patients received a second nasopharyngeal swab for SARS-Cov-2 independently from the trial, as required under the rules in force during the lockdown, to permit them to leave quarantine.

They were taught how to use the nebulizer and the doctor stayed for half an hour following the first treatment in the unlikely event of an allergic reaction. Patients were monitored closely for adverse events; there were no adverse effects at all, and all patients completed the full treatment. They were then followed for a month and all were free of symptoms or signs, recovering completely, except Patients 1 and 2, who felt fatigue (see Table 6). These two were also the oldest patients, 74 and 73 years respectively, and had co-morbidities, so would be regarded as very high risk.

### Dose and Method of Administration

1. Oral administration, 3 capsules/day: each capsule contained 300 mg of Theaphenon E (813 mg total catechins/day, 576 mg EGCG/day), for up to 15 days (ThE capsules, composition, see Table 5).
2. Nebulization of 5 ml of a 0.3% ThE solution made fresh daily in phosphate saline buffer at pH 5.8, with a breathing mask, twice a day for up to 15 days (total catechins: 27 mg/day; EGCG: 19/day, ThE powder composition, see Table 4).

This total daily dose of 595 mg is “below 800 mg EGCG” as suggested by the European Food Safety Authority (EFSA).

### Results

Clinical signs and symptoms were classic for all patients, such as cough and fever >38° C. (Table 6). Because of that, they all received a swab test and all were positive. They were visited by SC and enrolled in the study a median of 5 days following first swab test, range 3-6 days. Only PT 3 never had any clear symptom (see comments below), but nevertheless was found swab-positive. PT 1, 2, 5 and 8 were nested in familial clusters.

TABLE 6
Clinical conditions of the 10 patients of the trial at the doctor's first visit,
demographics, co-morbidities, and other medications including duration and dosage

| Patient | 1 | 2 | 3 | 4 | 5 | 6 | 7 | 8 | 9 | 10 |
|---|---|---|---|---|---|---|---|---|---|---|
| Symptoms | | | | | | | | | | |
| Difficulty breathing | X | X | | X | | | | | X | |
| Cough | X | X | | X | X | X | X | X | X | X |
| Tiredness 30 days after | X | X | | | | | | | | |
| No sense of smell | | | | | | X | | X | | |
| No sense of taste | | | | | | X | | X | | |
| Signs | | | | | | | | | | |
| Fever >38° C. | X | X | | X | X | X | X | X | X | X |
| Pneumonia TC score (%) | 10-15 | 10-15 | | 10-15 | | | | | 40 | |
| Hospitalised | No | No | No | No | No | No | No | No | Lo- | No |
| | | | | | | | | | flo O₂ | |
| Patient Demographics | | | | | | | | | | |
| Age | 74 | 73 | 50 | 53 | 47 | 27 | 55 | 28 | 66 | 38 |
| Gender | M | F | M | M | F | F | F | F | F | F |
| Comorbidities | X | X | | | | | | | X | |
| Other Treatments, dose | | | | | | | | | | |
| and duration | | | | | | | | | | |
| Steroids | X | X | | | | | | | | |
| | 10 days | 10 days | | | | | | | | |
| Azithromycin 500 mg | X | X | | X | | | | | X | |
| | 10 days | 10 days | | 4 days | | | | | 4 days | |
| Low molecular weight | X | X | | X | | | | | X | |
| heparin | 10 days | 10 days | | 10 days | | | | | 10 days | |
| Hydroxychloroquinone, | | | | | | | | | X | |
| 400 mg | | | | | | | | | 5 days | |
| Methylprednisolone | | | | X | | | | | | |
| 32 mg one shot | | | | 2 days | | | | | | |

All 10 evaluable patients recovered fully within treatment time, median 9.2 days, range 7-15 days. Seven out of 10 patients had a negative SARS-Cov-2 swab at a median of 8.7 days from starting therapy, range 6-13 days. Patients 3, 7 and 10 had a positive second swab at days 6, 5 and 6 respectively; all 3 were free of symptoms and fully recovered few days later, at the end of therapy, exiting quarantine. They did not infect anyone later on. The detailed laboratory results from the blood draws are shown in Table 7 (nb the columns of the table are split so that the table is presented in two parts).

Table 6 shows laboratory data results at recruitment (T0, first blood withdrawal: treatment begins) and at second blood withdrawal (T1, full recovery of patients). Bolded values are outside of normal range. SWAB result: result of first nasopharyngeal swab test (before treatment) and second test (during treatment): positive/negative; Patient 7 was positive at second test but with a very low load of virus nucleic acid. T0—2nd swab: elapsed time (days) from T0 to second swab (i.e. time of switch to negative swab during treatment for 7/10 patients); T0-T1: elapsed time from start of therapy to full recovery of patients (days). Statistical analysis by paired T-test (2-tails) shows statistical significance (p<0.05) for Eosin, AAT and CRP. Seven patients showed a decrease of 11-6 and ESR following treatment.

### Abbreviations in Table 7

PT Patient
White white blood cell count;
Hb haemoglobin;
Neutro neutrophils;
Lymph lymphocytes;
Mono mononuclear cells;
Eosin eosinophils;
ESR erythrocyte sedimentation rate;
AAT α-1 antitrypsin;
CRP C-reactive protein;
IL-6 interleukin-6 cytokine;
ND no data available.

TABLE 7
(part 1) - Laboratory Data Results

| | Blood | White ×10³/ | Hb | PLT ×10³/ | Neutro | Lymph | Mono | Eosin |
|---|---|---|---|---|---|---|---|---|
| PT | withdrawals | μL | g/dL | μL | % | % | % | % |
| 1 | T0 | 7.35 | 13.5 | 242 | 82.4 | 10.6 | 6.9 | 0.0 |
| | T1 | 6.77 | 13.3 | 179 | 86.1 | 9.4 | 4.1 | 0.2 |
| 2 | T0 | 6.06 | 13.7 | 295 | 62.4 | 27.1 | 9.2 | 0.8 |
| | T1 | 9.27 | 14.3 | 325 | 83.7 | 11.5 | 4.0 | 0.3 |
| 3 | T0 | 4.99 | 15.3 | 315 | 71.6 | 22.4 | 5.6 | 0.0 |
| | T1 | 5.03 | 14.3 | 254 | 51.4 | 38.4 | 7.8 | 1.4 |
| 4 | T0 | 8.33 | 14.9 | 269 | 73.8 | 19.6 | 4.7 | 1.7 |
| | T1 | 8.65 | 15.5 | 460 | 59.6 | 28.4 | 7.6 | 3.7 |
| 5 | T0 | 4.7 | 10.9 | 275 | 49.4 | 35.5 | 12.6 | 2.3 |
| | T1 | 9.3 | 10.4 | 395 | 54.7 | 33.9 | 8.6 | 2.6 |
| 6 | T0 | 6.63 | 13.8 | 197 | 41.8 | 48.0 | 9.4 | 0.3 |
| | T1 | 5.91 | 13.4 | 261 | 59.9 | 33.2 | 4.5 | 1.8 |
| 7 | T0 | 7.27 | 13.1 | 205 | 58.8 | 32.3 | 8.0 | 0.8 |
| | T1 | 8.13 | 13.6 | 265 | 63.3 | 29.4 | 6.2 | 0.7 |
| 8 | T0 | 7.12 | 12.3 | 277 | 61.5 | 25.6 | 9.6 | 2.7 |
| | T1 | 7.41 | 11.7 | 258 | 61.9 | 23.9 | 10.5 | 3.2 |
| 9 | T0 | 4.16 | 14.2 | 142 | 59.2 | 35.3 | 5.0 | 0.0 |
| | T1 | 7.63 | 12.5 | ND | 66.1 | 23.3 | 7.6 | 2.5 |
| 10 | T0 | 5.13 | 13.4 | 192 | 61.2 | 30.6 | 7.6 | 0.4 |
| | T1 | 6.26 | 13.0 | 210 | 65.3 | 28.1 | 5.9 | 0.5 |
| | Normal | 4-10 | 13.5-18.0 | 150-400 | 40-75 | 15-45 | 3-12 | 1-8 |
| | Range | | | | | | | |
| Mean | | 6.174 | 13.510 | 240.900 | 62.210 | 28.700 | 7.860 | 0.90 |
| T0 | | | | | | | | |
| Mean | | 7.414 | 13.278 | 289.667 | 65.200 | 25.950 | 6.680 | 1.69 |
| T1 | | | | | | | | |
| SD | | 1.377 | 1.256 | 54.836 | 11.705 | 10.226 | 2.448 | 1.68 |
| T0 | | | | | | | | |
| SD | | 1.530 | 1.500 | 89.202 | 11.326 | 9.353 | 2.125 | 0.90 |
| T1 | | | | | | | | |

TABLE 7
(part 2) - Laboratory Data Results

| | | | | | | | Time | |
|---|---|---|---|---|---|---|---|---|
| | | | | | | | T0-2^{nd} | Time |
| | Blood | ESR | AAT | CRP | IL-6 | Swab | swab | T0-T1 |
| PT | withdrawals | mm | mg/dL | mg/L | pg/L | result | (days) | (days) |
| 1 | T0 | 55 | 165 | 5.65 | 26.03 | POS | | |
| | T1 | 18 | 139 | 0.41 | 1.50 | NEG | 6 | 9 |
| 2 | T0 | 71 | 235 | 27.90 | 55.5 | POS | | |
| | T1 | 20 | 158 | 0.76 | 5.21 | NEG | 6 | 9 |
| 3 | T0 | 29 | 114 | 0.40 | 6.00 | POS | | |
| | T1 | 16 | 97 | 0.61 | 24.19 | POS | 6 | 9 |
| 4 | T0 | 38 | 170 | 23.00 | 69.08 | POS | | |
| | T1 | 55 | 155 | 6.33 | 9.90 | NEG | 10 | 7 |
| 5 | T0 | 74 | 166 | 15.00 | 8.25 | POS | | |
| | T1 | 38 | 114 | 0.61 | 2.87 | NEG | 13 | 15 |
| 6 | T0 | 22 | 207 | 3.72 | 7.82 | POS | | |
| | T1 | 19 | 230 | 6.54 | 4.34 | NEG | 10 | 12 |
| 7 | T0 | 6 | 120 | 3.72 | 184.95 | POS | | |
| | T1 | 10 | 116 | 0.42 | 3.00 | POS | 4 v. low | 7 |
| | | | | | | | viral load | |
| 8 | T0 | 6 | 127 | 1.00 | 12.75 | POS | | |
| | T1 | 2 | 81 | 0.39 | 12.75 | NEG | 6 | 9 |
| 9 | T0 | 78 | 254 | 18.00 | 124.42 | POS | | |
| | T1 | ND | ND | 13.30 | 14.76 | NEG | 10 | 8 |
| 10 | T0 | 34 | 151 | 0.53 | 17.37 | POS | | |
| | T1 | 21 | 136 | 0.29 | 194.05 | POS | 6 | 7 |
| | Normal | 2-25 | 90-200 | 0.001-5.000 | 0-10 | | | |
| | range | | | | | | | |
| Mean | | 41.30 | 70.90 | 9.89 | 51.22 | | 8.7 (only | 9.2 |
| T0 | | | | | | | swab NEG) | |
| Mean | | 22.11 | 136.22 | 2.97 | 7.26 | | 6-13 | 7-15 |
| T1 | | | | | | | | |
| SD | | 23.56 | 137.89 | 2.99 | 9.59 | | | |
| T0 | | | | | | | | |
| SD | | 41.30 | 170.90 | 9.89 | 51.22 | | | |
| T1 | | | | | | | | |

The results in Table 7 show that seven patients showed a decrease of IL-6, one did not change, only two increased. Among these two patients was PT 3: IL-6 increased in the blood test at 9 days; the earlier test results showed no eosinophils and high ESR, both markers for inflammation due to COVID-19; these values had improved to the normal range at 9 days. SC comments: “PT 3 is a mystery. He never had symptoms. PT 3 lives with the old mother in a small flat but she never had COVID. This patient may be a false positive case. PT 3 is still healthy, without symptoms, just like never had the disease.”

PT 7 had a high IL-6 of 185 pg/ml on the first lab test which returned to normal range at the second lab test 7 days later. The only other initial lab result was marginally low eosinophils. PT 7 had a very low viral load on the second swab, and was declared free of disease few days later, exiting quarantine.

PT 10, SC comments: “There is no explanation for the high IL-6 of PT 10 on day 7 (T1) or positive swab on day 6. At that time, PT 10 already had no symptoms and had recovered completely, exiting quarantine.”

IL-6 was the most informative in the blood tests. Seven of 10 patients had IL-6 greater than 10 pg/ml and 3/10 had greater than 100 pg/ml. The four oldest (PT 1, 2, 7 and 9) had high IL-6 values of 26, 55, 185 and 124 pg/ml respectively. These high values would normally indicate that these patients were developing SARS, so their rapid recovery is even more surprising.

There are many published studies on IL-6 as a prognosis for death or severe disease in COVID 19 patients. Values over 100 pg/ml have high relative risk (RR) or odds ratios (OR) for severe, critical disease or death. Del Valle et al measured cytokine values in 1,484 patients immediately on admission to Mount Sinai Health System, New York City. They determined a cutoff value of 70 pg/ml for IL-6 and tested it for prognosis of death and severity. Eight % of patients below the cutoff had died by 30 days, vs 15% of those above, Hazard Ratio (HR) 2.0, P<0.002. For disease severity, 76% of those below the cutoff sustained 02 saturation on room air >95%, vs 46% below the cutoff, HR 3.4, P<0.0021. (n=1,112) (see Del Valle et al) PT 4, 7 and 9 had IL-6 values of 69, 185 and 194 pg/ml respectively, yet they recovered in 7, 7 and 8 days respectively.

Considering other clinical parameters, 7/10 patients had elevated erythrocyte sedimentation rates (ESR) and 7/10 patients had normal rates by the second test. Seven out of 10 patients had reduced eosinophils, but 4 were only marginally low; of these 7, 4 patients had not returned to normal levels by the second blood test. Other lab values were normal for most patients.

Statistical analyses (paired T-test, two tails) showed a significant decrease of eosynophils, alpha-1 anti-trypsin and CRP following treatment.

All patients were free of symptoms and fully recovered by the time of second blood test, which was the primary objective. Median time to recovery was 9.2 days (range 7-15) and 70% had a negative swab. These results are fully documented with official lab records.

Such rapid recovery from COVID-19 is rare. Mancuso et al followed 1,162 patients with a positive swab in Reggio Emilia province, Italy, from 26 February to 22 March; the province of Reggio Emilia is next to the province of Parma. In their study the median time to a first negative swab was 31 days from first positive swab (IQR 24-41), estimated by the Kaplan Meier method. At 10 days, only 0.56% of these patients had a first negative swab. Under local regulations, only symptomatic patients were tested.

In our study, 70% of patients had a first negative swab at a median of 8.7 days, range 6-13 days. The null hypothesis is that the rate of negative testing is unaffected by green tea catechins and follows the same distribution as the population of Reggio Emilia. The probability this would happen by chance is very small.

### Example 4—Preparation of Two Components of a Medical Device

The base gel (first component) was obtained by dissolving in water polyvinyl alcohol of a molecular weight equal to or close to 90,000 in a concentration of 1% w/w. The solution was stirred until obtaining a homogeneous solution followed by adding high molecular weight sodium alginate at a concentration of 0.5% w/w. An ascorbic acid/sodium ascorbate buffer at a concentration of 0.2% w/w was added to the whole, and finally a mixture of epicatechin, epigallocatechin, epicatechin-3-gallate and epigallocatechin-3-gallate (or other polyphenols or flavonoids) was added at a concentration of 0.3% w/w. The salt solution of the second component is represented by a one-molar aqueous solution of calcium chloride.

### Example 5—Application of the Two Components of Example 4

The gel prepared according to Example 1 is applied by spray to oral and nasal mucous membranes, and then sprayed with a calcium chloride solution prepared according to Example 1, which also preferably should be administered by means of a spray nebulizer. In this context, it should be pointed out that the subject matter of the present description also relates to a kit comprising at least one container for the first component, and at least one container for the second component, both preferably provided with a nebulizer/spray system, including use of the kit in the prophylactic and therapeutic treatment of coronavirus pathologies and specifically COVID-19.

### Example 6—Alternative Preparation of the Two Components of a Medical Device

The base gel (first component) was obtained by dissolving polyvinyl alcohol with a molecular weight of 90,000 and a concentration of 1% w/w in water. The solution was stirred until obtaining a homogeneous solution, followed by adding sodium alginate with a specific viscosity of 4,000 cP and a concentration of 0.5% w/w. An ascorbic acid/sodium ascorbate buffer at a concentration of 0.2%, acetylsalicylic acid at a concentration of 5% w/w, and finally a mixture of epicatechin, epigallocatechin, epicatechin-3-gallate and epigallocatechin-3-gallate (or other polyphenols or flavonoids) at a concentration of 0.3% w/w was added.

The cross-linking agent (second component) for transforming the gel into a solid with the consistency of a soft rubber was an aqueous solution of 1 molar calcium chloride.

### Example 7—Alternative Preparation and Administration of Two Components of a Medical Device

The ingredients of the two components of the product were as set out below. High molecular weight sodium alginate was obtained from Carlo Erba Reagents S.A.S.

| | Ingredient | Amount | |
|---|---|---|---|
| Component 1 | Deionized water (non- | 1.00 | L |
| | pyrogenic, grade III) | | |
| | Methyl paraben | 1.8 | g |
| | Propyl paraben | 0.6 | g |
| | High molecular weight | 5.0 | g |
| | sodium alginate (medical | | |
| | grade) | | |
| | Theaphenon ® E | 3.0 | g |
| | Sodium ascorbate (food or | 2.0 | g |
| | medical grade) | | |
| | Hydrochloric acid (1M | 3.3 | mL |
| | aqueous solution) | | |
| Component 2 | Water | 1.00 | L |
| | Calcium chloride | 110.98 | g |

(Food/medical grade)

### Preparation of Component 1

Methyl paraben and propyl paraben were dissolved in deionized water. The temperature of the solution was adjusted to 30° C. and high molecular weight sodium alginate was added with vigorous stirring, followed by gentle stirring for 1 hour. The temperature of the solution was maintained at 30° C. and Theaphenon® E and sodium ascorbate were dissolved with gentle stirring. Finally, 1M hydrochloric acid was added to the solution at a temperature of between ambient temperature and 30° C.

### Preparation of Component 2

In order to prepare the second component, calcium chloride was dissolved in deionized water.

The components are packaged into separate containers, each of which is provided with a metered volume spray nozzle, which administers a volume of 0.1 to 0.5 mg per spray.

The product may be provided with instructions for use which direct a patient to apply the product as follows:
Shake the container containing Component 1 and apply 2-4 sprays on to the oral or nasal mucosa;
Shake the container containing Component 2 and apply 1-2 sprays on top of Component 1;
Reapply every 8 hours or after eating or drinking.

The containers may contain about 20-50 mL of Component 1 or Component 2 and may provide a dose of about 0.1 to 0.5 mL or Component 1 or Component 2 per spray. The exact volume dispensed per spray may be adjusted depending on the required dose of Theaphenon® E

### REFERENCES

Eui Hong Byun, Yoshinori Fujimura, Koji Yamada and Hirofumi Tachibana (May 2010). “TLR4 Signaling Inhibitory Pathway Induced by Green Tea Polyphenol Epigallocatechin-3-Gallate through 67-kDa Laminin Receptor”. J Immunol 2010; 185:33-45.
Eui-Hong Byun, Toshinori Omura, Koji Yamada, Hirofumi Tachibana (2011). “Green tea polyphenol epigallocatechin-3-gallate inhibits TLR2 signaling induced by peptidoglycan through the polyphenol sensing molecule 67-kDa laminin receptor”. FEBS Letters 2011; 585: 814-820.
Del Valle, D. M.; Kim-Schulze, S.; Huang, H. H.; Beckmann, N. D.; Nirenberg, S.; Wang, B.; Lavin, Y.; Swartz, T. H.; Madduri, D.; Stock, A.; et al. An inflammatory cytokine signature predicts COVID-19 severity and survival. Nat. Med. 2020, 26, 1636-1643, doi:10.1038/s41591-020-1051-9.
Forni D, Cagliani R, Clerici M, Sironi M (January 2017). “Molecular Evolution of Human Coronavirus Genomes”. Trends in Microbiology. 25 (1): 35-48.
Grant M C, Geoghegan L, Arbyn M, Mohammed Z, McGuinness L, Clarke E L, Wade R G (23 Jun. 2020). “The prevalence of symptoms in 24,410 adults infected by the novel coronavirus (SARS-CoV-2; COVID-19): A systematic review and meta-analysis of 148 studies from 9 countries”. PLOS ONE. 15 (6): e0234765.
Henry, B. M.; De Oliveira, M. H. S.; Benoit, S.; Plebani, M.; Lippi, G. Hematologic, biochemical and immune biomarker abnormalities associated with severe illness and mortality in coronavirus disease 2019 (COVID-19): A meta-analysis. Clin. Chem. Lab. Med. 2020, 58, 1021-1028.
Mancuso, P.; Venturelli, F.; Vicentini, M.; Perilli, C.; Larosa, E.; Bisaccia, E.; Bedeschi, E.; Zerbini, A.; Rossi, P. G. Temporal profile and determinants of viral shedding and of viral clearance confirmation on nasopharyngeal swabs from SARS-CoV-2-positive subjects: a population-based prospective cohort study in Reggio Emilia, Italy. BMJ Open. 2020, 10:e040380. doi: 10.1136/bmjopen-2020-040380.
Mehta, P.; McAuley, D. F.; Brown, M.; Sanchez, E.; Tattersall, R. S.; Manson, J. J. COVID-19: consider cytokine storm syndromes and immunosuppression. Lancet 2020, 395, 1033-1034
Menegazzi, M.; Campagnari, R.; Bertoldi, M.; Crupi, R.; Di Paola, R.; Cuzzocrea, S. Protective effect of epigallocatechin-3-gallate (EGCG) in diseases with uncontrolled immune activation: Could such a scenario be helpful to counteract COVID-19? Int. J. Mol. Sci. 2020, 21, 5171.
Mhatre, S.; Srivastava, T.; Naik, S.; Patravale, V. Antiviral activity of green tea and black tea polyphenols in prophylaxis and treatment of COVID-19: A review. Phytomedicine 17 Jul. 2020.
Ohishi, T.; Goto, S.; Monira, P.; Isemura, M.; Nakamura, Y. Anti-inflammatory Action of Green Tea. Antiinflamm. Antiallergy. Agents Med. Chem. 2016, 15, 74-90, doi:10.2174/1871523015666160915154443.
Yeong-Seon Won, Motofumi Kumazoe, Kanako Takamatsu, Yuki Shinoda, Saki Sonoda, Kenji Okada, Takehisa Okamoto, Hirofumi Tachibana (August 2016). “Green tea cultivar ‘Benifuuki’ potentiates split vaccine-induced immunoglobulin A production”. J Nat Med (2017) 71: 68-75

1. A method for the treatment or prevention of coronavirus infection, the method comprising administering to a patient in need of such treatment an effective amount of a catechin-containing composition comprising a catechin selected from epigallocatechin-3-gallate (EGCG), epigallocatechin (EGC), epicatechin-3-gallate (ECG) and epicatechin (EC) or a mixture thereof.

2. The method according to claim 1 wherein the catechin-containing composition comprises EGCG and EGC.

3. The method according to claim 1, wherein the catechin-containing composition is a green tea extract and comprises catechins in an amount of 75 to 98% by weight with respect to the dry weight of the composition.

4. The method according to claim 1, wherein the catechin-containing composition comprises EGCG in an amount of 50% to 80% by weight with respect to the dry weight of the composition.

5. The method according to claim 4, wherein the catechin-containing composition comprises EGC in an amount of 5% to 20% by weight with respect to the dry weight of the composition.

6. The method according to claim 4, wherein the composition comprises 0 to 1% caffeine, 0 to 1% theobromine and 0 to 1% gallic acid by weight with respect to the dry weight of the composition.

7. (canceled)

8. The method according to claim 1, wherein the coronavirus infection is SARS-CoV-2 infection.

9. A method according to claim 1 wherein the catechin-containing composition is comprised in a pharmaceutical composition, wherein the pharmaceutical composition is adapted for oral administration; for administration by inhalation; or for administration to the oral or nasal mucosa.

10. (canceled)

11. The method according to claim 9 wherein the pharmaceutical composition comprises capsules for oral administration or a solution for inhaled administration via a nebulizer.

12.-17. (canceled)

18. The method according to claim 11 wherein the pharmaceutical composition is administered to a patient in an amount such that the dose of the catechin-containing composition is from 10 mg to 3000 mg per day.

19. A method according to claim 18, wherein the pharmaceutical composition is administered orally in an amount such that the dose of the catechin-containing composition is from 300 mg to 2000 mg per day, suitably 600 mg to 1800 mg per day; or
wherein the pharmaceutical composition is administered by inhalation to a patient in an amount such that the dose of the catechin-containing composition is from 10 mg to 60 mg per day, suitably from 25 mg to 35 mg per day.

20.-21. (canceled)

22. A medical device which is adapted for administration to the oral or nasal mucosa and which comprises a first component and a second component
characterized in that:
the first component is an aqueous gel which comprises a biocompatible polymer, a polyacid and/or salt thereof, a preservative and a catechin-containing composition comprising a catechin selected from epigallocatechin-3-gallate (EGCG), epigallocatechin (EGC), epicatechin-3-gallate (ECG) and epicatechin (EC) or a mixture thereof; and
the second component is an aqueous solution comprising a salt of a bivalent, trivalent or multivalent cation, or a combination of such salts.

23. A medical device according to claim 22, wherein the biocompatible polymer is polyvinyl alcohol (PVA) having a molecular weight of 10,000 to 1,000,000 and wherein the PVA is present in an amount of 1% to 4% by weight of the first component.

24. A medical device according to claim 22, wherein the polyacid or salt thereof is sodium alginate and wherein the sodium alginate is present in an amount of 0.001% to 5% by weight of the first component.

25. A medical device according to claim 22, wherein the catechin-containing composition is present in the first component in an amount such that the first component comprises 0.01% to 5% by weight, suitably 0.1 to 0.5% by weight of EGCG, EGC, ECG, EC or a mixture thereof.

26. A medical device according to claim 22, wherein the first component further comprises an anti-inflammatory agent e.g. selected from acetylsalicylic acid, flurbiprofen or combinations thereof e.g. in an amount of 0.001% to 30% by weight of the first component and/or an anti-viral agent e.g. (2S)-2-{(2R, 3S, 4R, 5R)-[5-(4-Aminopyrrole [2,1-f][1,2,4] triazin-7-yl)-5-cyano-3, 4-dihydroxy-tetrahydrofuran-2-ylmethoxy] phenoxy-(S)-phosphorylamine} propionic acid 2-ethyl-butyl ester (remdesivir) e.g. in an amount of 0.001% to 30% by weight of the first component.

27. A medical device according to claim 22, wherein the second component is an aqueous solution comprising a chloride or iodide salt.

28. A medical device according to claim 27, wherein the second component is an aqueous solution of calcium chloride, magnesium chloride or zinc chloride at a concentration of from 0.001 M to saturation.

29. A medical device according to claim 22, wherein the first and second components are to be administered in the form of a spray, suitably a nebulised spray.

30. A bioadhesive film situated at the oral and/or nasal mucous membranes of a patient and obtainable by applying to the oral and/or nasal mucous membranes of a patient the first and second components of the medical device according to claims 22.

31. A kit comprising a first container containing the first component of the medical device according to claim 22, and a second container containing the second component of the medical device.

32. The kit of claim 31 wherein each of the first and second containers is provided with a nebuliser/spray system and where the nebuliser/spray system of each container comprises a means for administering a metered volume of the first and second components.

33.-34. (canceled)

35. A method for the treatment or prevention of coronavirus infection, the method comprising administering to a patient in need of such treatment or prevention an effective amount of the components of a medical device according to claims 22.

36. (canceled)

37. A method for the treatment or prevention of lung injury, the method comprising administering to a patient in need of such treatment an effective amount of a catechin-containing composition comprising a catechin selected from epigallocatechin-3-gallate (EGCG), epigallocatechin (EGC), epicatechin-3-gallate (ECG) and epicatechin (EC) or a mixture thereof.

38. (canceled)

39. The method according to claim 37 wherein the lung injury is caused by coronavirus (e.g. SARS-CoV2) infection or
wherein the lung injury is caused by asthma, chronic obstructive pulmonary disease or fibrosis.

40. (canceled)

41. An aqueous gel which comprises a biocompatible polymer, a polyacid and/or salt thereof, a preservative and a catechin-containing composition comprising a catechin selected from epigallocatechin-3-gallate (EGCG), epigallocatechin (EGC), epicatechin-3-gallate (ECG) and epicatechin (EC) or a mixture thereof.
